(19) United States
(12) Patent Application Publication
(10) Pub. No.: US 2026/0083397 A1
(43) Pub. Date: Mar. 26, 2026

(54) Implant Communication

(71) Applicant: Howmedica Osteonics Corp., Mahwah, NJ (US)

(72) Inventors: Andrew Meyer, Davie, FL (US); Carlos O. Alva, Boynton Beach, FL (US); Chad Bean, Norton, MA (US); Wael Hazin, Plantation, FL (US)

(21) Appl. No.: 18/894,285

(22) Filed: Sep. 24, 2024

(51) Int. Cl. A61B 5/00; A61B 5/01; A61F 2/38

(52) U.S. Cl. CPC A61B 5/4851; A61B 5/01; A61B 5/686; A61F 2/38; A61B 2560/0209; A61B 2560/0214; A61B 2560/045; A61B 2560/0462; A61B 2562/0219; A61B 2562/0223; A61B 2562/0247; A61B 2562/0252; A61B 2562/0271

(57) ABSTRACT

Disclosed herein is an implant with sensors that can communicate with an external source under predetermined conditions. The implant can include at least one sensor configured to detect implant data, a power source, an activation switch in communication with the at least one sensor, the activation switch configured to receive an external input, and a communication module configured to wirelessly communicate with an external source. The implant data can be any of an implant condition or adjacent surgical site condition. The activation switch can be configured to initiate wireless communication between the communication module and the external source to transmit the implant data in response to receiving the external input.

### FIELD OF INVENTION

The present disclosure relates to an implant with sensors, and particularly to an implant with sensors that can communicate with an external source under predetermined conditions.

### BACKGROUND OF THE INVENTION

Implantable medical devices, particularly smart implants—i.e., implants with sensors, encounter challenges in power management due to the constricted confines available for battery integration and the inherent spatial limitations. A smart implant is generally designed to achieve exceptional energy efficiency to prolong battery life, ensuring sustained operation over prolonged durations, which could extend to several years. This requirement becomes even more critical as the smart implant may need to periodically engage in wireless communication with an external source for data transmission and device configuration, amplifying the challenges associated with preserving battery power.

The process of sending complex implant sensor data, especially at high frequencies or over extended distances, may require substantial energy. Moreover, the need for secure data transmission protocols to protect sensitive health information introduces additional layers of complexity and energy demand. These security measures, while crucial for patient privacy and data integrity, further strain the device's limited energy resources. Maintaining a wireless transceiver of the smart implant in an active state for continuous communication can lead to exorbitant energy consumption rates which could dramatically diminishing the smart implant's operational lifespan from the anticipated years to merely days or weeks.

Therefore, there exists a need for implants with sensors capable of efficient communication with an external source.

### BRIEF SUMMARY OF THE INVENTION

Disclosed herein are implants with sensors that can communicate with an external source under predetermined conditions and related methods for such communication.

In accordance with an aspect of the present disclosure, an implant is provided. An implant according to this aspect may include at least one sensor configured to detect implant data, a power source, an activation switch in communication with the at least one sensor, the activation switch configured to receive an external input, and a communication module configured to wirelessly communicate with an external source. The implant data may be any of an implant condition or adjacent surgical site condition. The activation switch may be configured to initiate wireless communication between the communication module and the external source to transmit the implant data in response to receiving the external input.

Continuing in accordance with this aspect, the activation switch may be configured to initiate wireless communication between the communication module and the external source in response to receiving the external input when the external input corresponds to a predetermined value.

Continuing in accordance with this aspect, the activation switch may be configured to initiate wireless communication between the communication module and the external source in response to receiving the external input when the external input corresponds to a predetermined pattern.

Continuing in accordance with this aspect, the external input may be any of a vibration or acoustic input. The vibration input may be any of a vibration frequency, a vibration magnitude, and a vibration duration. The acoustic input may be any of an acoustic frequency, an acoustic magnitude, and an acoustic duration.

Continuing in accordance with this aspect, the external input may be generated by the external source.

Continuing in accordance with this aspect, the external input may be generated by an external device. The external device may be a smartphone.

Continuing in accordance with this aspect, the at least one sensor may be any of a pH sensor, a temperature sensor, a pressure sensor, a load sensor, an accelerometer, a gyroscope, an IMU and a Hall sensor operatively coupled to a controller of the implant. The controller may be a microcontroller in communication with the activation switch and the communication module.

Continuing in accordance with this aspect, the adjacent surgical site condition may include any of a temperature, pressure, and pH.

Continuing in accordance with this aspect, the implant may be a joint implant. The joint implant may be a knee implant including a tibial component and a femoral component, the at least one sensor, the power source, the activation switch, and the communication module may be disposed within the tibial component.

Continuing in accordance with this aspect, the power source may be a battery.

Continuing in accordance with this aspect, the activation switch may include a filter configured to compare the external input with any of a predetermined value and a predetermined pattern.

Continuing in accordance with this aspect, the communication module may be a receiver configured to wirelessly transmit the implant data to the external source.

Continuing in accordance with this aspect, the communication module may be a transceiver configured to wirelessly transmit the implant data to the external source and receive external data from the external source.

Continuing in accordance with this aspect, the external source may any of a smartphone, computer, tablet, and a network.

Continuing in accordance with this aspect, the activation switch may be configured to transition the implant from a low-power mode to a high-power mode when the external input corresponds to a predetermined value or predetermined pattern. The implant may consume greater energy from the power source in the high-power mode relative to the low-power mode.

In accordance with another aspect of the present disclosure, an implant is provided. An implant according to this aspect may include at least one sensor configured to detect implant data, the implant data may be any of an implant condition or adjacent surgical site condition, a power source, and an activation switch in communication with the at least one sensor. The activation switch may be configured to receive an external input. The activation switch may be configured to transition the implant from a first operational state to a second operational state in response to receiving the external input.

Continuing in accordance with this aspect, the first operational state may be a low-powered mode, and the second operational state may be high-power mode. The implant may consume greater energy from the power source in the high-power mode relative to the low-power mode.

Continuing in accordance with this aspect, a first sensor of the implant may be activated in the first operational state and a second sensor of the implant may be activated in the second operational state.

Continuing in accordance with this aspect, the activation switch may be configured to transition the implant from the first operational state to the second operational state in response to receiving the external input when the external input corresponds to a predetermined value.

Continuing in accordance with this aspect, the activation switch may be configured to transition the implant from the first operational state to the second operational state in response to receiving the external input when the external input corresponds to a predetermined pattern.

Continuing in accordance with this aspect, the external input may be any of a vibration or acoustic input. The vibration input may be any of a vibration frequency, a vibration magnitude, and a vibration duration. The acoustic input may be any of an acoustic frequency, an acoustic magnitude, and an acoustic duration.

Continuing in accordance with this aspect, the external input may be generated by an external device. The external device may be a smartphone.

Continuing in accordance with this aspect, the at least one sensor may be any of a pH sensor, a temperature sensor, a pressure sensor, a load sensor, an accelerometer, a gyroscope, an IMU and a Hall sensor operatively coupled to a controller of the implant.

Continuing in accordance with this aspect, the adjacent surgical site condition may include any of a temperature, pressure, and pH.

Continuing in accordance with this aspect, the implant may be a joint implant. The joint implant may be a knee implant including a tibial component and a femoral component, the at least one sensor, the power source. The activation switch may be disposed within the tibial component.

Continuing in accordance with this aspect, the power source may be a battery. Continuing in accordance with this aspect, the activation switch may include a filter configured to compare the external input with any of a predetermined value and a predetermined pattern.

### BRIEF DESCRIPTION OF THE DRAWINGS

A more complete appreciation of the subject matter of the present disclosure and the various advantages thereof can be realized by reference to the following detailed description, in which reference is made to the following accompanying drawings:

FIG. 1 is a schematic view of an implant according to an embodiment of the present disclosure;

FIG. 2 is a flowchart showing steps of an implant communication according to an embodiment of the present disclosure;

FIG. 3 is a flowchart showing steps of an implant communication according to an embodiment of the present disclosure;

FIG. 4 is a flowchart showing steps of an implant communication according to another embodiment of the present disclosure;

FIG. 5 is a graph showing an acceleration input pattern according to an embodiment of the present disclosure, and

FIG. 6 is a flowchart showing steps of an implant operation according to an embodiment of the present disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

The term “smart implant” means an implant with at least one sensor. The term “joint implant” means a joint implant system comprising two or more implants. It should be understood that the term “implant performance” as used herein includes implant condition and related patient condition such as the condition of tissue and bone around the implant, etc.

As used herein, the terms “implant” and “smart implant” will be used interchangeably and as such, unless otherwise stated, the explicit use of either term is inclusive of the other term. The terms “power” and “energy” will be used interchangeably and as such, unless otherwise stated, the explicit use of either term is inclusive of the other term.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior”means closer to the head, and the term “inferior”means more distant from the head.

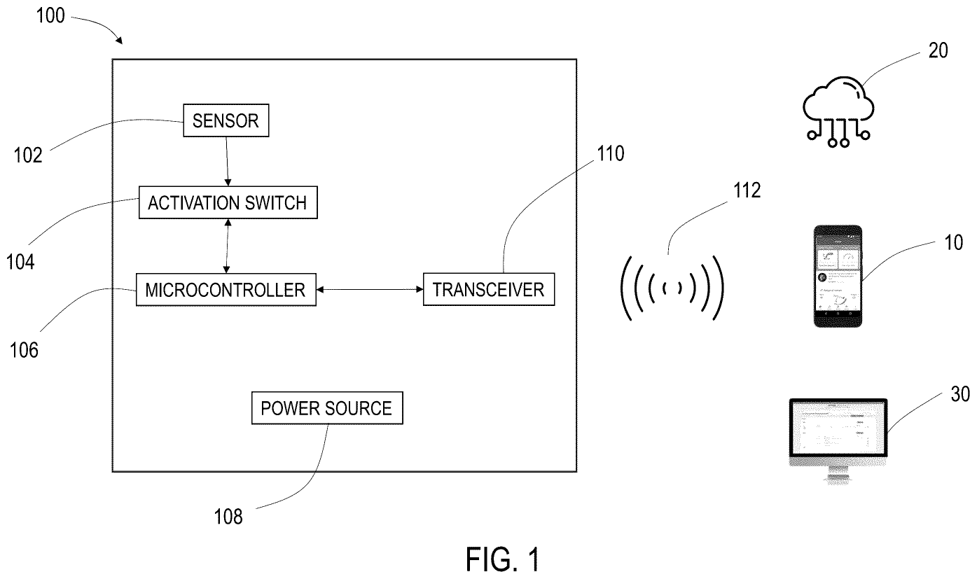

FIG. 1 is a schematic view of an implant 100 according to an embodiment of the present disclosure. Implant 100 includes at least one sensor 102. Sensor 102 can take numerous forms depending on the functional requirements of the implant. Examples of sensor 102 can include, but are not limited to, a load sensor capable of monitoring mechanical stresses, a temperature sensor for detecting thermal variations, a pressure sensor responsive to fluidic or gaseous pressures, an accelerometer for measuring changes in velocity or orientation, a Hall sensor for detecting magnetic fields, a gyroscope for orientation and rotational speed detection, a magnetometer for magnetic intensity measurement, a pH sensor to gauge the acidity or basicity of surrounding tissues or fluids, and an inertial measurement unit (IMU) that integrates multiple sensing capabilities. Sensor 102 can perform a breadth of functions such as measuring, detecting, and comparing various aspects of the implant's performance and its interaction within the surgical site. Taking a knee implant as an example, sensor 102 can be a load sensor specifically designed to accurately measure and record the dynamic loading forces exerted on and by the knee joint during patient activity. Implant 100 can include multiple sensors, each of a different type, to provide a comprehensive monitoring system capable of tracking a range of physiological and biomechanical parameters simultaneously.

Implant 100 includes a power source 108 such as a battery to power the sensor and other electronic components of implant 100. Power source 108 can be an integrated power source 108, such as a rechargeable lithium-ion battery or other suitable energy storage component configured to supply an appropriate and sustained level of electrical energy to various operative elements within implant 100, including but not limited to, sensor 102, control circuitry, and communication apparatus.

A transceiver 110 of implant 100 allows the implant to communicate wirelessly 112 with an external source such as a smartphone 10, cloud 20, computer 30, etc. Transceiver 110 serves a dual purpose within the architecture of implant 100. On one hand, it enables the transmission of collected sensor data and operational status updates from the implant to external devices. On the other hand, it facilitates the reception of incoming instructions or configuration adjustments from an array of external sources. Wireless communication 112 can be realized by the inclusion of a dedicated data transmission element, such as an antenna (not shown). The design and positioning of the antenna within implant 100 are optimized for both the propagation of signals through the human body and minimal interference with surrounding tissues and other medical devices.

Various wireless protocols can be used for the bi-directional communication between implant 100 and the external source including near field communication (“NFC”), Bluetooth Low Energy (BLE), Medical Implant Communication Service (MICS), Wi-Fi, Z-Wave, etc. The selection of a particular wireless protocol for the communication between implant 100 and the external source can be determined by several parameters. One such parameter is the required communication range-different environments and use-cases may dictate different optimal distances for uninterrupted and reliable data transmission. Additionally, the volume and frequency of the data exchange can be considered. Certain clinical applications may necessitate the transfer of large data sets or frequent transmission of sensor readings, thereby requiring a more robust protocol that can manage higher bandwidths and provide greater data throughput. The chosen communication protocol can provide strong encryption and secure connection capabilities to ensure patient confidentiality and integrity of sensitive medical information. Factors such as energy efficiency of the implant's power source, the feasibility of recharging the power source, and the overall power consumption of the communication protocol can influence the protocol selection to preserve the implant's functional longevity and minimize the need for invasive maintenance procedures.

Wirelessly connecting implant 100 to an external source allows the implant to transfer sensor data to the external source and to receive instructions from the external device to change or optimize implant performance if necessary. Ensuring consistent relay of sensor data is contingent upon several factors including patient compliance. This process enables healthcare professionals (HCPs) or surgeons to consistently monitor the status of the implant, such as a knee joint implant, and respond promptly to indicators of malfunction or atypical recovery patterns. However, maintaining such vigilance may prove burdensome for patients. Patients may encounter difficulties with the technological aspects of the monitoring process, such as managing and remembering complex login credentials or navigating the interface of the external device, which can lead to underutilization or neglect. Additionally, powering the implant's sensor array and computational unit demands a battery that not only fits within the spatial constraints of the implant but also has ample energy capacity to ensure sustained functionality. It is imperative for the battery to be durable, stable, and safe within the physiological environment. To facilitate data transfer, implant 100 enters an “advertising mode,” a state where it seeks out and establishes a connection with compatible wireless networks such as Bluetooth Low Energy (BLE). However, being in this mode continuously can lead to accelerated battery depletion, posing a challenge for the energy management system within the implant.

Activation switch 104 of implant 100, as illustrated in FIG. 1, is designed to enhance the authentication and connectivity process between the implant and an external source while effectively managing battery energy consumption. Activation switch 104 is equipped with a motion filter, which may include a vibration or sound filter, aimed at conserving power by enabling wireless communications only when specific vibration or sound inputs are detected. The activation switch is configured to identify particular vibration or sound patterns, serving as a trigger to initiate bi-directional data transmission between the implant and the external source.

Sensor 102 can be programmed to monitor for a pattern of vibrations or sounds, which may vary in parameters such as frequency, magnitude, and duration, and may include intervals or pauses between vibrations. During operation, a patient can position smartphone 10 or another vibrational device on or near the skin where the sensor has been implanted. The vibrational device then emits the predefined sequence of vibrations or sounds, which travel through the patient's body to reach the implant. Activation switch 104 within the implant records and identifies the vibrational signal, initiating the wake-up process once the signal is recognized. If communication is successfully established, data transmission begins otherwise, the device returns to its low-power operating mode, awaiting the next authentication attempt.

As shown in FIG. 1 and more fully described below activation switch 104 receives sensor data from sensor 102 and initiates bi-directional communication with an external source by activating transceiver 110 via a microcontroller 106. Activation switch 104 is configured to function as a sentinel, receiving a stream of sensor data from sensor 102, which is actively monitoring various parameters such as load, temperature, pressure, or motion within the context of the implant's environment. Upon the acquisition of relevant sensor data, activation switch 104 is programmed to detect predefined vibration or sound patterns or thresholds, which, once recognized, trigger the device to pivot from a passive state into an active one, engaging its communication capabilities. Microcontroller 106 can be a specialized and integrated circuit within implant 100 to receive a signal from activation switch 104 and, in response, commands transceiver 110 to initiate bi-directional wireless communication with a designated external source. Thus, transceiver 110 serves as the communicative bridge between implant 100 and the outside world, operates in an optimally timed fashion to exchange vital data between the implant and external devices such as clinicians'monitoring systems, patient interfaces, or medical databases.

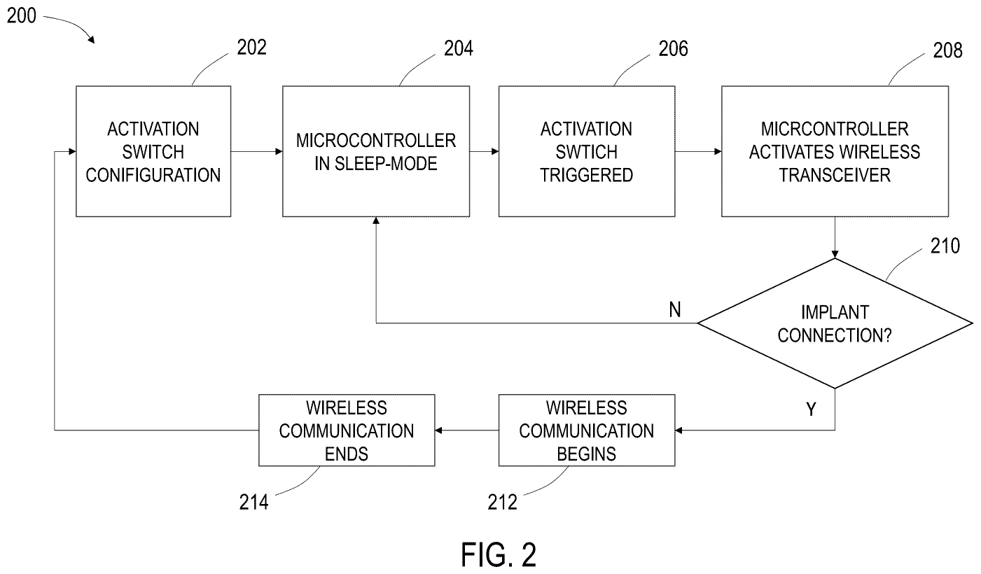

FIG. 2 is a flowchart illustrating the steps involved in implant communication 200 utilizing an activation switch, in accordance with an embodiment of the present disclosure. These steps are described in relation to the components of implant 100, which in this embodiment, is exemplified by a knee joint implant. The process begins with a setup step 202, where the microcontroller 106 is employed to configure the activation switch 104 with specific vibration or sound filters.

During the setup, the microcontroller 106 can be programmed to select or define the parameters of the vibration or sound filters, including for example, the frequency, amplitude, and duration of the signals that the activation switch 104 will recognize. The configuration may also involve setting thresholds for these parameters to ensure that the implant responds only to particular patterns of vibration or sound that match the predefined criteria. Additionally, the setup can include adjustments to account for potential environmental noise or body-specific characteristics, ensuring the filter is tuned to accurately detect the intended input signals.

For example, the vibration filter may be configured to respond to a sequence of pulses at a specific frequency range, while the sound filter might be set to recognize audio signals within a particular decibel level and pitch. These filters ensure that the implant remains in a low-power state until the correct trigger is detected, at which point the microcontroller activates the communication process, allowing for secure data transmission between the implant and the external device.

In a step 204, microcontroller 106 transitions into a “sleep mode,” a state designed to substantially reduce the implant's energy consumption while allowing activation switch 104 to remain operational with minimal current draw to conserve energy further. The activation switch 104 is equipped with a sound and vibration filter, which monitors for specific acoustic or vibrational inputs that match predefined characteristics. When the identified sound or vibration pattern is detected, the activation switch 104 activates microcontroller 106 in step 206. This activation shifts microcontroller 106 from its energy-saving sleep mode and triggers transceiver 110 to awaken and prepare for data communication processes.

Transceiver 110 is activated for a predetermined duration, sufficient to establish a connection with an external source. If the connection is not successfully made within this time frame, microcontroller 106 returns to its energy-conserving sleep mode in a step 210, thus preserving the implant's battery life. Conversely, upon successful connection, a bi-directional communication channel is established between implant 100 and the external source for wireless communication in a step 212.

Data transmitted from implant 100 may include sensor readings, sensor analytics, and other implant-related metrics, while incoming data from the external source could consist of operational commands, such as sensor calibration updates, adjustments to sensor operational timings, recommendations for sensor data storage levels, and more. Additionally, the calibration parameters of the sound and vibration filter associated with activation switch 104 can be dynamically updated, incorporating new reference signals, dynamic ranges, and acoustic or vibrational patterns. These updates enhance the responsiveness and effectiveness of the implant's interaction with the user and the surrounding environment.

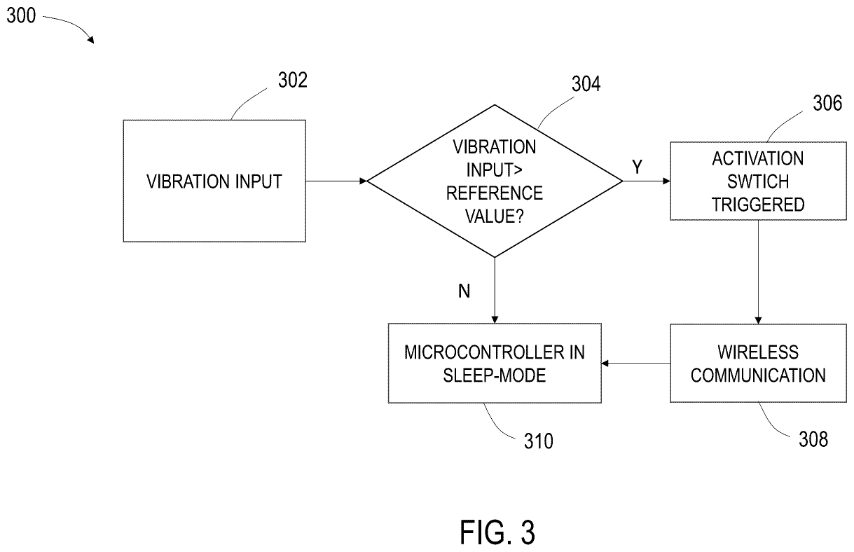

Referring now to FIG. 3, there is shown a flowchart illustrating the steps involved in implant communication 300 using a sound and/or vibration filter according to an embodiment of the present disclosure. The sound or vibration filter is designed to detect specific acoustic or vibrational inputs that trigger the communication data transfer process. The methodology for setting and updating the vibration or sound threshold, which serves as a reference value 304, can be defined during the initialization of implant 100, such as during the power-up phase. Alternatively, the reference value 304 may be recalibrated or reset upon the completion of a communication transmission, thereby establishing a new benchmark for future sound or vibration detection events. Additionally, reference value 304 can be programmed to automatically re-establish itself at regular intervals, such as every second, creating a dynamic reference point that requires sensor deviations within the predefined window to activate communication.

Reference value 304 can take on various forms. For instance, sensor 102 may continuously monitor for a specific vibrational frequency, acoustic signal, or combination thereof, sustained for a particular duration and above a certain magnitude, using measurement devices such as a microphone or an inertial measurement unit (IMU). In operation, a patient can hold a phone or another device on or near the skin where the sensor has been implanted. The external device would then emit the desired vibrational or acoustic signal at the specified frequency. The signal travels through the patient's body to the implanted device, where it is recorded and recognized by the sensor, triggering the wake-up process in a step 306.

Once the communication is initiated, if the connection is successfully established, the implant will proceed with the data transfer. If not, the device returns to its low-power operating mode in step a 310, conserving energy. For example, if a sensor is implanted in a patient's knee, it can be configured to detect a frequency of 261 Hz±5 Hz, corresponding to middle C on a piano, sustained for 3 seconds. The patient could place their knee in proximity to a piano and play the note for the required duration, prompting the implant to wake up and begin advertising for a wireless connection to an external transceiver, such as a phone.

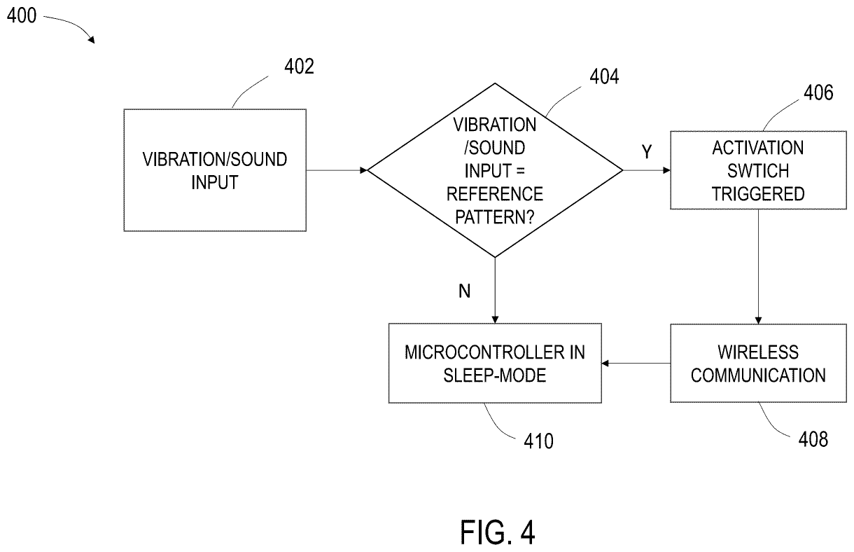

FIG. 4 is a flowchart illustrating the steps involved in an implant communication 400, utilizing a vibration and/or sound filter, according to another embodiment of the present disclosure. Implant communication 400 is similar to implant communication 300, with corresponding steps referred to by similar numerals within the 400-series. However, in this embodiment, instead of relying on a single reference value, implant communication 400 utilizes a reference pattern 404. This reference pattern consists of a specific sequence of vibrations or acoustic inputs that the implant sensor is programmed to recognize. When an input pattern matches this reference pattern, the activation switch is triggered in a step 406 to initiate communication.

The implant sensor can be configured to search for this pattern either continuously or at specific intervals, which could be dictated by time, external cues such as the location of the external device relative to the implant. The reference pattern can include various parameters such as frequency, magnitude, duration of individual signals, and potentially pauses or delays between successive vibrations or sounds. This configuration allows the implant to remain in a low-power mode until the exact pattern is detected, thus optimizing battery life.

A patient can hold a phone or another vibrational device on or near the skin where the sensor has been implanted. The external device would then emit the predefined sequence of vibrations or sounds that travel through the patient's body to the implant. The implant's sensor, upon detecting and matching the input with the reference pattern 404, triggers the wake-up process in a step 406. This process initiates communication by activating the implant's microcontroller and transceiver, allowing for a connection to be established with an external device in a step 408.

The external vibrational device, such as a phone, can emit the desired sequence of vibrations or sounds, which will propagate through the patient's body to the implant. The implant device records and analyzes the incoming signal, comparing it against the stored reference pattern. If a match is found, the wake-up process begins, and the implant transitions from low-power mode to active communication mode. If the connection is successfully established, the device proceeds with data transmission; otherwise, it returns to its low-power operating mode to conserve energy.

For example, a knee sensor can be configured to listen for a specific pattern such as five pulses at a frequency of 60 Hz, each pulse sustained for 240 milliseconds, with a 160-millisecond delay between each pulse. The patient would hold their phone against their thigh and use an application to initiate the connection to the implant device. The phone would emit five pulses according to the predefined pattern, which the implant sensor would detect. Upon recognizing the pattern, the sensor triggers the device to begin advertising for a wireless connection to the phone. The phone then connects to the sensor, and the app notifies the patient that the connection has been successfully established.

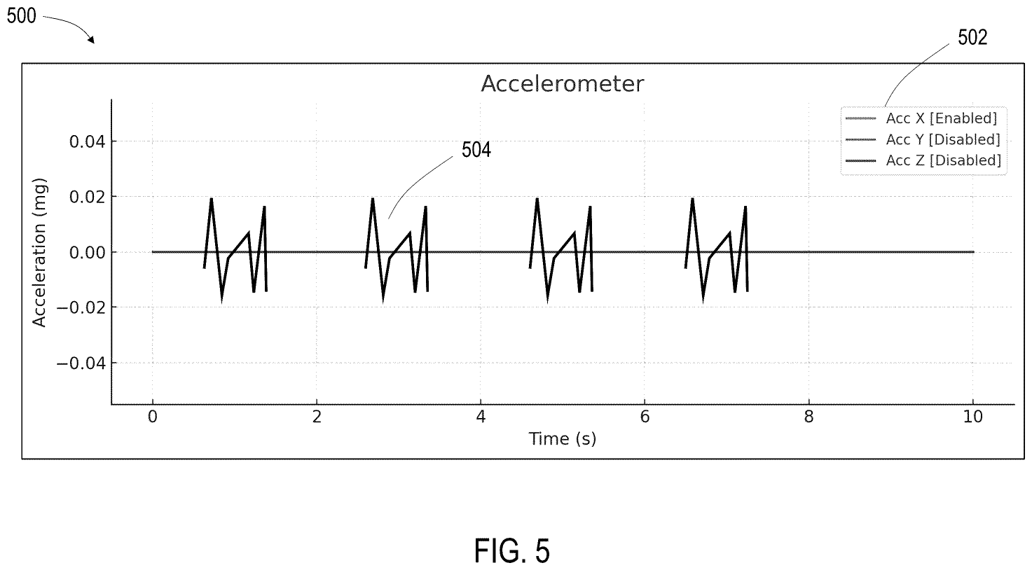

FIG. 5 shows a graph 500 illustrating patterns detected by an accelerometer within the implant sensor according to an embodiment of the present disclosure. Graph 500 measures acceleration over time depicting acceleration data captured across one or more axes (X, Y, and Z) as shown in 502. Pattern 504 represents a sequence of acceleration events that repeats at regular intervals. When the implant's sensor detects that this pattern repeats consistently, it confirms a match with the reference value stored within the system, thereby triggering the communication process in implant communication 400.

In this embodiment, the accelerometer data is used to identify specific vibration and/or acoustic patterns emitted from the external device. If the sensor detects that this acceleration pattern 504 matches the predefined reference value, the implant transitions from a low-power state to an active communication mode, allowing data to be transmitted to an external device, such as a smartphone or a wearable monitor. While the use of an accelerometer to measure acceleration is disclosed in this embodiment, other embodiments may utilize different types of measurements to achieve similar outcomes.

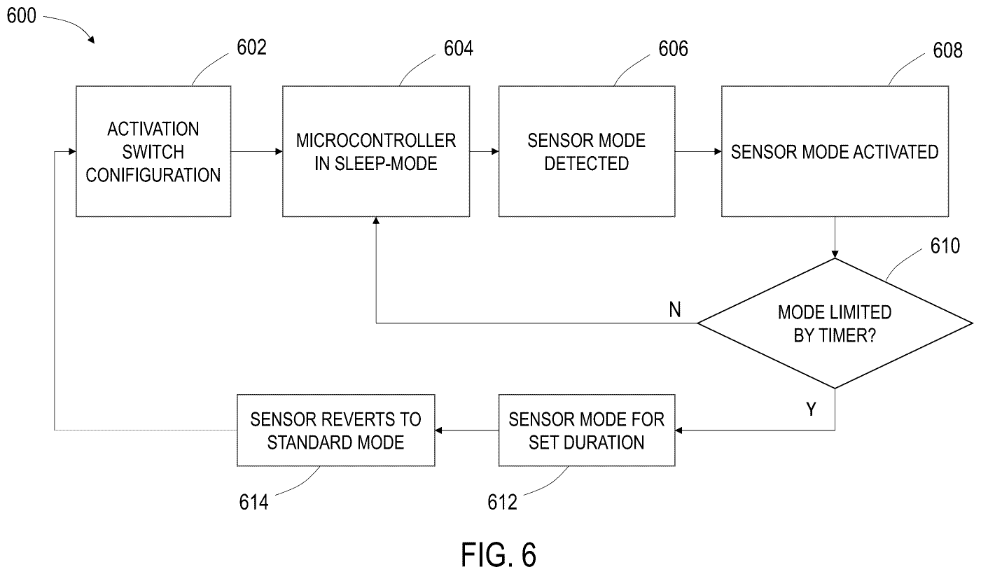

Referring now to FIG. 6, a flowchart showing steps of an implant operation 600 according to one embodiment of the present disclosure is shown. Implant operation can control the various operational modes of the implant device. This operation can include the capability to adjust the power settings of the implant, which may involve running the implant at reduced or increased power levels depending on the desired functionality, such as acquiring additional sensor readings or enhancing transmission capabilities. For implants equipped with multiple types of sensors, the operation may involve managing the utilization of these sensors at different times to optimize performance or conserve power. Implant operation 600 shares some similarities with the implant communication process previously disclosed. However, in this instance, the focus is not on communication wake-up but on altering the operational mode of the sensors, effectively modifying the overall functionality of the implant. The operation is triggered by a vibration or acoustic switch filter that detects either a specific threshold or pattern in a step 606, as previously described. Upon detection and matching of this pattern or threshold in a step 606, the implant initiates various sensor modes in step 608. These modes may be time-limited, with the implant reverting to its original or another predetermined condition once the specified time period has elapsed (steps 610 and 612). Alternatively, the sensor mode could be activated and adjusted continuously based on real-time vibration or acoustic filter readings.

The implant operation described here functions similarly to the previously discussed implant communication process, with the key difference being that, in this context, the focus is on changing sensor modes rather than initiating communication wake-up. The sensor within the implant is configured to detect for one or more specific frequencies or patterns. Depending on the detected frequency or pattern, the sensor will switch to an appropriate operational mode. These modes may include, but are not limited to: a deep sleep mode, in which many functions are disabled to conserve power; a high-frequency data collection mode, where sensors gather data at an increased rate compared to standard operating modes; a specific sensor collection mode, where certain sensors, such as an IMU, are disabled to save power, while others, like Hall sensors, remain active; and an advertising mode, where the implant seeks to establish a wireless connection with another device, such as a smartphone. Each operational mode is associated with a distinct pattern that activates it, and, if necessary, a corresponding pattern may be used to revert the implant to its standard operating mode. In some implementations, a simple timer could be employed to automatically return the implant to its standard operating mode after a set duration.

For example, where a sensor is implanted into a person's knee-the sensor can be configured to enter a deep sleep mode upon receiving three pulses at 80 Hz, transmitted through the skin via a smartphone. Several weeks later, when a clinician needs to collect data, the patient places the smartphone on their thigh, and the device transmits a three-second vibration at 100 Hz, prompting the sensor to switch to standard data collection mode. Subsequently, a week later, the patient, guided by an application, places the smartphone on their thigh again, which then transmits five pulses at 60 Hz to activate the advertising mode. In this mode, the smartphone connects to the sensor and offloads the collected data to cloud storage, where the clinician can access and analyze the information.

It should be understood that the implants disclosed herein are not limited to a singular mode of communication with an external device. Implant 100 can be configured to incorporate multifaceted communication strategies, either as standalone options or in synergy with the activation switch to enhance the functional connectivity between the implant and external devices for improved performance and user experience. In some embodiments, implant 100 can selectively initiate communication with the external device based on spatial parameters. Correspondingly, the implant can be programmed to recognize and respond to the proximity of an external source. Communication is thus not solely contingent on the input signals but can also be activated if the external source is within a pre-established spatial threshold, a predetermined distance from the implant. This geospatial criteria ensure that the implant interacts purposefully with authorized and nearby devices, enhancing security while also conserving system resources.

While a knee joint implant is disclosed above, all or any of the aspects of the present disclosure can be used with any other implant such as a hip implant, shoulder implant a spinal implant, an intramedullary nail, a bone plate, a bone screw, an external fixation device, an interference screw, etc. Although, the present disclosure generally refers to implants, the systems and method disclosed above can be used with trials to provide real time information related to trial performance. Sensor shape, size and configuration can be customized based on the type of implant and patient-specific needs.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. An implant comprising:
at least one sensor configured to detect implant data, the implant data being any of an implant condition or adjacent surgical site condition;
a power source;
an activation switch in communication with the at least one sensor, the activation switch configured to receive an external input; and
a communication module configured to wirelessly communicate with an external source,
wherein the activation switch is configured to initiate wireless communication between the communication module and the external source to transmit the implant data in response to receiving the external input.

2. The implant of claim 1, wherein the activation switch is configured to initiate wireless communication between the communication module and the external source in response to receiving the external input when the external input corresponds to a predetermined value.

3. The implant of claim 1, wherein the activation switch is configured to initiate wireless communication between the communication module and the external source in response to receiving the external input when the external input corresponds to a predetermined pattern.

4. The implant of claim 1, wherein the external input is any of a vibration or acoustic input.

5. The implant of claim 4, wherein the vibration input is any of a vibration frequency, a vibration magnitude and a vibration duration.

6. The implant of claim 4, wherein the acoustic input is any of an acoustic frequency, an acoustic magnitude and an acoustic duration.

7. The implant of claim 1, wherein the external input is generated by the external source.

8. The implant of claim 1, wherein the external input is generated by an external device.

9. The implant of claim 8, wherein the external device is a smartphone.

10. The implant of claim 1, wherein the at least one sensor is any of a pH sensor, a temperature sensor, a pressure sensor, a load sensor, an accelerometer, a gyroscope, an IMU and a Hall sensor operatively coupled to a controller of the implant.

11. The implant of claim 10, wherein the controller is a microcontroller in communication with the activation switch and the communication module.

12. The implant of claim 1, wherein the adjacent surgical site condition includes any of a temperature, pressure, and pH.

13. The implant of claim 1, wherein the implant is a joint implant.

14. The implant of claim 13, wherein the joint implant is a knee implant including a tibial component and a femoral component, the at least one sensor, the power source, the activation switch, and the communication module being disposed within the tibial component.

15. The implant of claim 1, wherein the power source is a battery.

16. The implant of claim 1, wherein the activation switch includes a filter configured to compare the external input with any of a predetermined value and a predetermined pattern.

17. The implant of claim 1, wherein the communication module is a receiver configured to wirelessly transmit the implant data to the external source.

18. The implant of claim 1, wherein the communication module is a transceiver configured to wirelessly transmit the implant data to the external source and receive external data from the external source.

19. The implant of claim 1, wherein the external source is any of a smartphone, computer, tablet, and a network.

20. The implant of claim 1, wherein the activation switch is configured to transition the implant from a low-power mode to a high-power mode when the external input corresponds to a predetermined value or predetermined pattern, the implant consuming greater energy from the power source in the high-power mode relative to the low-power mode.
